(19) United States
(12) Patent Application Publication
(10) Pub. No.: US 2019/0038308 A1
(43) Pub. Date: Feb. 7, 2019

(54) JAW MEMBERS FOR SURGICAL INSTRUMENTS AND SURGICAL INSTRUMENTS INCORPORATING THE SAME

(71) Applicant: Covidien LP, Mansfield, MA (US)

(72) Inventors: JOHN P. ROBB, BROOMFIELD, CO (US); JAMES S. CUNNINGHAM, BOULDER, CO (US); DAREN T. FAYE, LONGMONT, CO (US); DYLAN S. HOBACK, ARVADA, CO (US)

(21) Appl. No.: 16/008,369

(22) Filed: Jun. 14, 2018

Related U.S. Application Data
(60) Provisional application No. 62/540,102, filed on Aug. 2, 2017.

(51) Int. Cl. A61B 17/32

(52) U.S. Cl. CPC A61B 17/320092; A61B 2017/320074; A61B 2017/320094

(57) ABSTRACT

A jaw member includes a body having an elongated distal portion extending distally from a proximal flange portion. The elongated distal portion includes a floor and a pair of sidewalls that cooperate to define a cavity having a first width extending between the sidewalls. One or more engagement features extends inwardly from one of the sidewalls into the cavity such that the cavity defines a reduced width at a location of the engagement feature. A jaw liner is slidable into the cavity to engage the jaw liner with the structural body. The jaw liner defines an at-rest width less than or equal to the first width and greater than the reduced width such that, upon slidable insertion of the jaw liner into the cavity, the jaw liner is compressed at the location and retained therein via an interference-fit engagement. Surgical instruments including the jaw member are also provided.

### CROSS REFERENCE TO RELATED APPLICATION

The present application claims the benefit of and priority to U.S. Provisional Application Ser. No. 62/540,102, filed on Aug. 2, 2017 the entire contents of which are incorporated herein by reference.

### BACKGROUND

### Technical Field

The present disclosure relates to surgical instruments and, more particularly, to jaw members of energy-based surgical instruments and energy-based surgical instruments incorporating the same.

### Background of Related Art

Many energy-based surgical instruments employ an end effector including one or more jaw members configured to facilitate clamping, manipulating, and/or applying energy to tissue to treat tissue.

Ultrasonic surgical instruments, for example, utilize ultrasonic energy in the form of ultrasonic vibrations to coagulate, cauterize, fuse, seal, cut, desiccate, fulgurate, or otherwise treat tissue. The ultrasonic energy is typically produced by a generator and transducer and is transmitted along a waveguide to an end effector of the ultrasonic surgical instrument. The end effector may include a blade that receives the ultrasonic energy from the waveguide for application to tissue and a jaw member configured to clamp tissue between the blade and the jaw member to facilitate treatment thereof.

As vibrations induced in the blade and thermal energy generated by the blade may be transferred to the jaw member, jaw members have been developed that include a jaw liner engaged to a structural body of the jaw member. However, a need still exists to provide a jaw member having a jaw liner engaged to the structural body in such a way that the jaw member is both effective and not overly difficult or expensive to manufacture.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

Provided in accordance with aspects of the present disclosure is a jaw member for a surgical instrument. The jaw member includes a structural body and a jaw liner. The structural body includes a proximal flange portion and an elongated distal portion extending distally from the proximal flange portion. The elongated distal portion includes a floor and a pair of opposed sidewalls that cooperate to define a cavity having a first width extending between the opposed sidewalls. The elongated distal portion further includes one or more engagement features extending inwardly from one of the opposed sidewalls into the cavity such that the cavity defines a reduced width at a location of the engagement feature. The jaw liner is configured for slidable insertion into the cavity to engage the jaw liner with the structural body. The jaw liner defines an at-rest width that is less than or equal to the first width and greater than the reduced width such that, upon slidable insertion of the jaw liner into the cavity, the jaw liner is compressed at the location of the engagement feature and retained therein via an interference-fit engagement.

In an aspect of the present disclosure, the cavity includes a relatively wide base portion and a relatively narrow body portion cooperating to define a generally T-shaped cross-sectional configuration of the cavity. In such aspects, the first width may be defined within the relatively wide base portion of the cavity, and the engagement feature may extend inwardly into the relatively wide base portion of the cavity.

In another aspect of the present disclosure, the jaw liner includes a relatively wide base portion and a relatively narrow body portion that cooperate to define a generally T-shaped cross-sectional configuration of the jaw liner.

In another aspect of the present disclosure, the jaw liner is formed from a compliant material such as, for example, PTFE.

In still another aspect of the present disclosure, the cavity defined within the structural body includes an open proximal mouth to enable distal sliding of the jaw liner through the open proximal mouth and into a central portion of the cavity. The structural body may additionally or alternatively include a distal lip configured to inhibit further distal sliding of the jaw liner once the jaw liner is fully disposed within the cavity.

In yet another aspect of the present disclosure, the engagement feature is a wedge. The wedge, more specifically, may include a proximally-facing angled surface and a distally-facing perpendicular surface.

In still yet another aspect of the present disclosure, the engagement feature is a hump.

In another aspect of the present disclosure, a first plurality of spaced-apart engagement features extends inwardly from one of the opposed sidewalls into the cavity and a second plurality of spaced-apart engagement features extends inwardly from the other of the opposed sidewalls into the cavity. In such aspects, the first and second pluralities of spaced-apart engagement features are arranged in a staggered configuration relative to one another.

A surgical instrument provided in accordance with aspects of the present disclosure includes a handle assembly, an elongated body portion extending distally from the handle assembly, and an end effector operably disposed at a distal end portion of the elongated body portion. The end effector assembly includes an energy-delivering component and a jaw member positioned to oppose the energy-delivering component. The jaw member is movable relative to the energy-delivering component between an open position and a closed position for clamping tissue between the jaw member and the energy-delivering component. The jaw member may further include any or all of the features of the jaw members detailed above, other otherwise described herein.

In an aspect of the present disclosure, the energy-delivering component is an ultrasonic blade and the elongated body portion includes an ultrasonic waveguide extending therethrough. The ultrasonic waveguide is configured to transmit ultrasonic energy to the ultrasonic blade. In such aspects, the jaw liner, in the closed position of the jaw member, is configured to oppose the ultrasonic blade.

### BRIEF DESCRIPTION OF THE DRAWINGS

The above and other aspects and features of the present disclosure will become more apparent in view of the following detailed description when taken in conjunction with the accompanying drawings, wherein:

FIG. 1 is a perspective view of an ultrasonic surgical instrument including an end effector disposed in an open condition;

FIG. 2A is an enlarged, perspective view of a distal end portion of the ultrasonic surgical instrument of FIG. 1A, wherein the end effector is disposed in the open condition and wherein a distal portion of the outer sleeve is removed to illustrate the components hidden thereby;

FIG. 2B is an enlarged, perspective view of the distal end portion of the ultrasonic surgical instrument of FIG. 1A, wherein the end effector is disposed in a clamping condition;

FIG. 3 is an exploded, perspective view of a jaw member of the end effector of the ultrasonic surgical instrument of FIG. 1;

FIG. 4A is a top, longitudinal, cross-sectional view of a structural body of the jaw member of FIG. 3;

FIG. 4B is a top, longitudinal, cross-sectional view of the jaw member of FIG. 3 including a jaw liner engaged with the structural body;

FIG. 4C is a transverse, cross-sectional view of the structural body of the jaw member of FIG. 3;

FIG. 4D is a transverse, cross-sectional view of the jaw liner of the jaw member of FIG. 3;

FIG. 4E is a transverse, cross-sectional view of the jaw member of FIG. 3;

FIG. 5A is a top, longitudinal, cross-sectional view of a structural body of another jaw member configured for use with the end effector of the ultrasonic surgical instrument of FIG. 1; and

FIG. 5B is a top, longitudinal, cross-sectional view of the structural body of FIG. 5A including a jaw liner engaged therewith.

### DETAILED DESCRIPTION

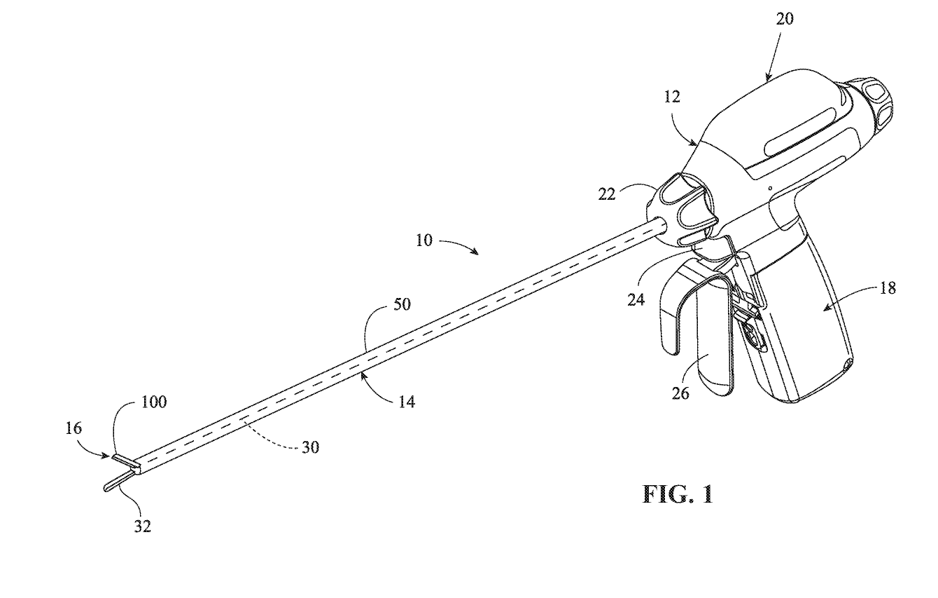

Jaw members and surgical instruments including such jaw members are provided in accordance with the present disclosure and detailed hereinbelow. Referring to FIG. 1, although the jaw members of the present disclosure are described for use with an ultrasonic surgical instrument 10, the jaw members of the present disclosure may alternatively be configured for use with any other suitable surgical instrument, including ultrasonic surgical instruments different from ultrasonic surgical instrument 10.

Ultrasonic surgical instrument 10 generally includes a handle assembly 12, an elongated body portion 14, and an end effector 16. Handle assembly 12 supports a power supply, e.g., a battery assembly 18, and an ultrasonic transducer and generator assembly (“TAG”) 20, although ultrasonic surgical instrument 10 may alternatively be configured as a tethered instrument wherein the power supply and generator are remote components coupled to handle assembly 12 via one or more surgical cables (not shown). Handle assembly 12 includes a rotation wheel 22, an activation button 24, and a clamp trigger 26. Battery assembly 18 and TAG 20 are each releasably coupled to handle assembly 12 and are removable therefrom to facilitate disposal of any disposable components, e.g., handle assembly 12, elongated body portion 14, and/or end effector 16, and reprocessing of any reusable components, e.g., battery assembly 18 and TAG 20.

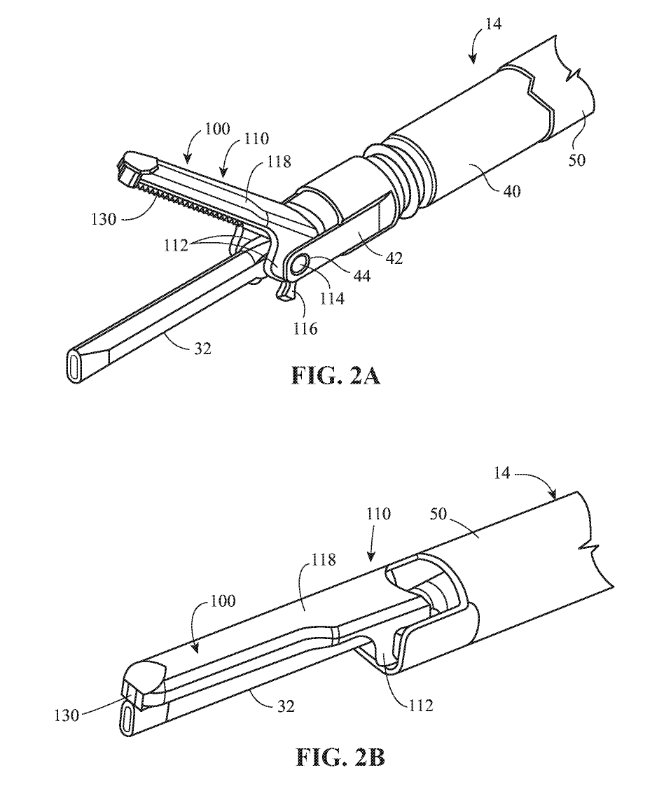

With additional reference to FIGS. 2A and 2B, elongated body portion 14 includes a waveguide 30 which extends from handle assembly 12 to end effector 16. A blade 32 of end effector 16 extends distally from waveguide 30. A proximal end portion of waveguide 30 is configured to engage the ultrasonic transducer of TAG 20 to enable the transmission of ultrasonic energy along waveguide 30 from the ultrasonic transducer of TAG 20 to blade 32.

Elongated body portion 14 further includes an inner tube 40 disposed about waveguide 30 and extending between handle assembly 12 and end effector 16. Inner tube 40, more specifically, includes a proximal end portion that extends into handle assembly 12. Inner tube 40 further includes a distal end portion including a pair of spaced-apart support arms 42 (only one of which is illustrated in FIG. 2A), each defining a transverse aperture 44 therethrough. Apertures 44 are configured to pivotably receive pivot bosses 114 of proximal flanges 112 of structural body 110 of jaw member 100 of end effector 16 to pivotably engage jaw member 100 to inner tube 40 at the distal end portion thereof.

An outer tube 50 is slidably disposed about inner tube 40 and similarly extends between handle assembly 12 and end effector 16. Outer tube 50, more specifically, includes a proximal end portion that extends into handle assembly 12 and operably couples to clamp trigger 26 by way of a drive assembly (not shown), and a distal end portion defining a cut-out (not shown) that operably receives legs 116 of proximal flanges 112 of structural body 110 of jaw member 100. As a result of this configuration, clamp trigger 26 may be manipulated between an un-actuated position and an actuated position to translate outer tube 50 between an advanced position and a retracted position, thereby pivoting jaw member 100 between an open position (FIGS. 1 and 2A), wherein jaw member 100 is spaced-apart from blade 32, and a closed position (FIG. 2B), wherein jaw member 100 is approximated relative to blade 32. Alternatively, the arrangement of inner and outer tubes 40, 50, respectively, may be reversed.

Referring to FIG. 1, rotation wheel 22 is operably coupled to waveguide 30, inner tube 40, and outer tube 50 such that rotation of rotation wheel 22 relative to handle assembly 12 similarly rotates waveguide 30, inner tube 40, and outer tube 50 relative to handle assembly 12, thereby also rotating blade 32 and jaw member 100 relative to handle assembly 12 in a similar manner. Activation button 24 is configured to selectively activate battery assembly 18 and TAG 20 to produce ultrasonic energy that is transmitted along waveguide 30 to blade 32 of end effector 16.

As detailed above, jaw member 100 includes a structural body 110 including a pair of proximal flanges 112 which enable pivotable coupling of jaw member 100 with inner tube 40 and operable coupling of jaw member 100 with outer tube 50. Structural body 110 of jaw member 100 further includes an elongated distal portion 118 extending distally from the pair of proximal flanges 112. A jaw liner 130 is engaged with elongated distal portion 118 of structural body 110 and is positioned to oppose blade 32 such that blade 32 is inhibited from contacting structural body 110 of jaw member 100. As a result, the transfer of ultrasonic vibrations and/or thermal energy from blade 32 to structural body 110 during use is reduced.

With general reference to FIGS. 1-2B, in use, ultrasonic instrument 10 is advanced into a surgical site and manipulated such that end effector 16 is positioned with tissue to be treated disposed between jaw member 100 and blade 32 with jaw member 100 disposed in the open position (FIGS. 1 and 2A). Thereafter, clamp trigger 26 is squeezed towards battery assembly 18 from the un-actuated position to the actuated position to translate outer tube 50 about inner tube 40 and relative to end effector 16, thereby pivoting jaw member 100 relative to blade 32 from the open position to the closed position to clamp tissue between jaw member 100 and blade 32 and, more specifically, between jaw liner 130 of jaw member 100 and blade 32. Blade 32 may then be activated, e.g., via depression of activation button 24, to supply ultrasonic energy from TAG 20, along waveguide 30, to blade 32. The ultrasonic energy provided at blade 32 is used to treat, e.g., coagulate, cauterize, fuse, seal, cut, desiccate, fulgurate, etc., tissue clamped between jaw member 100 and blade 32.

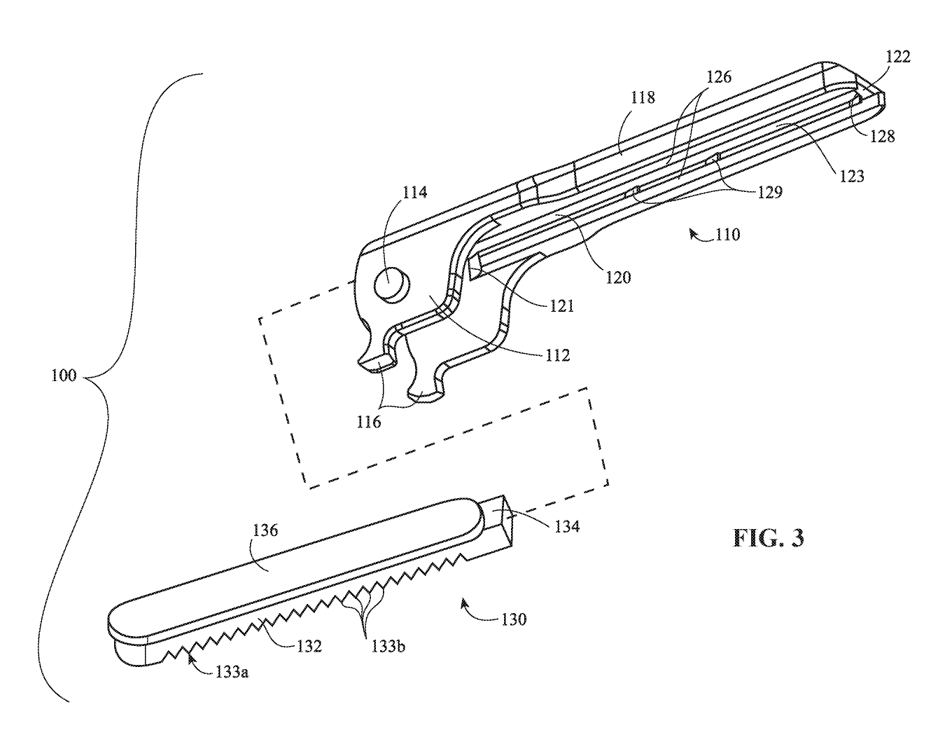

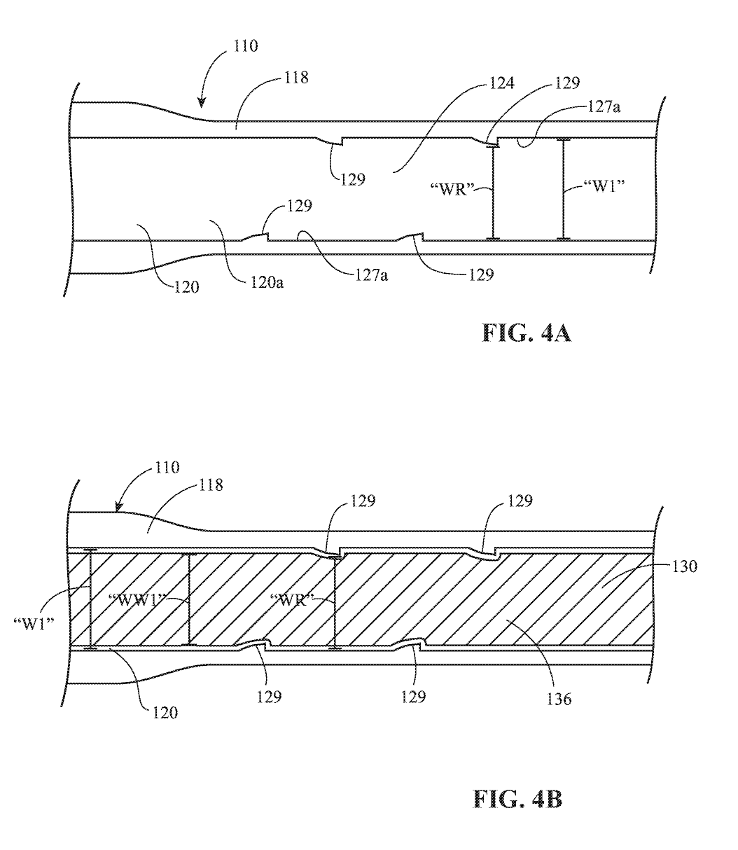

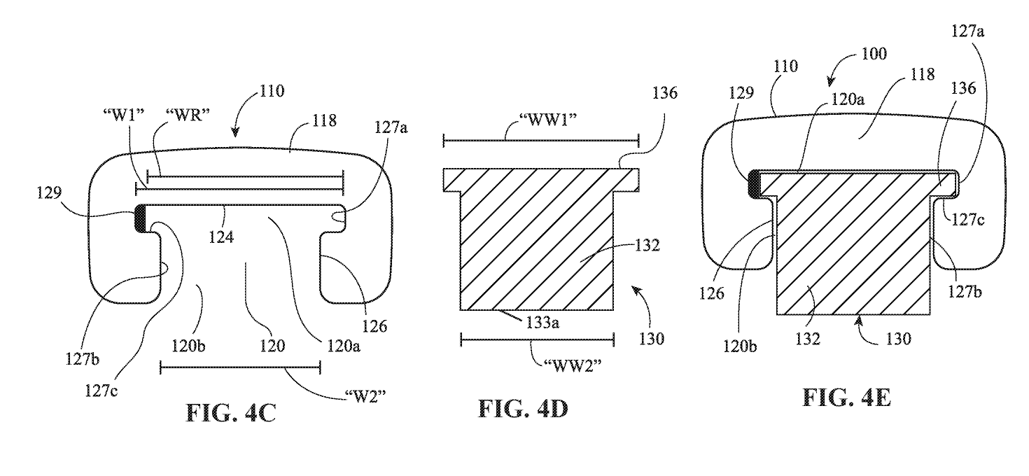

With reference to FIGS. 3-4E, jaw member 100 is described in greater detail. Jaw member 100, as noted above, includes a structural body 110 and a jaw liner 130. As also noted above, structural body 110 includes proximal flanges 112 and elongated distal portion 118 extending distally from the pair of proximal flange 112. Structural body 110 is relatively rigid to provide integrity and support to jaw member 100 to enable jaw member 100 to apply sufficient clamping pressure to tissue grasped between jaw member and an opposing structure, e.g., blade 32 (FIGS. 1-2B). Structural body 110 may be fabricated from a metal, for example, stainless steel, or any other suitable material, and may be monolithically formed via machining, stamping, metal injection molding, or may be formed in any other suitable manner via any other suitable process.

As also noted above, each proximal flange 112 of structural body 110 of jaw member 100 includes a pivot boss 114 (only one of which is illustrated in FIG. 3) configured to enable pivotable coupling of jaw member 100 to inner tube 40 (FIG. 2A) and a leg 116 configured to enable operable coupling of jaw member 100 to outer tube 50 (FIG. 2B). Other suitable pivot and/or drive couplings are also contemplated.

Elongated distal portion 118 of structural body 110 of jaw member 100, as noted above, extends distally from the pair of proximal flanges 112. Although illustrated as defining a linear configuration, it is also contemplated that elongated distal portion 118 define a curved configuration. More specifically, elongated distal portion 118 may curve laterally towards one of the proximal flanges 112 (and, thus, away from the other proximal flange 112), or may curve upwardly away from or downwardly towards blade 32 (FIGS. 1-2B).

Continuing with reference to FIGS. 3-4C, elongated distal portion 118 of structural body 110 defines a cavity 120 extending longitudinally therealong that is configured to receive jaw liner 130 therein. Cavity 120 defines an open proximal mouth 121, an open distal neck 122, and a central portion 123 extending between open proximal mouth 121 and open distal neck 122. Central portion 123 of cavity 120 is defined by an interior floor 124 of elongated distal portion 118 of structural body 110 and opposing sidewalls 126 of elongated distal portion 118 of structural body 110. Each Sidewall 126 includes a first sidewall section 127a, a second sidewall section 127b, and a shoulder 127c defined between the respective first and second sidewall sections 127a, 127b. First sidewall sections 127a of opposing sidewalls 126 are positioned adjacent interior floor 124 and are further-spaced apart from one another as compared to second sidewall sections 127b. As a result, cavity 120 defines a generally T-shaped cross-sectional configuration including a relatively wide base portion 120a having a width “W1” and a relatively narrow body portion 120b has a width “W2.” Both base portion 120a and body portion 120b of cavity 120 communicate with open proximal mouth 121 of cavity 120, while only body portion 120b of cavity 120 communicates with open distal neck 122. Base portion 120a of cavity 120 terminates in a distal lip 128 adjacent the distal end portion of elongated distal portion 118 of structural body 110.

Elongated distal portion 118 of structural body 110 of jaw member 100 further includes a plurality of engagement features, in the form of wedges 129, disposed on and extending inwardly from one or both of first sidewall sections 127a of sidewalls 126. Wedges 129 are oriented such that the angled surfaces thereof are disposed in a generally proximally-facing orientation and such that the perpendicular surfaces thereof are disposed in a generally distally-facing orientation, although other configurations are also contemplated. A plurality of spaced-apart wedges 129 may extend inwardly from the first sidewall section 127a of each sidewall 126 in a staggered arrangement, as illustrated in FIG. 4A, although other configurations are also contemplated. With wedges 129 protruding inwardly from first sidewall sections 127a into base portion 120a of cavity 120, base portion 120a of cavity 120 defines a reduced width “WR” at the location of each wedge 129.

Referring still to FIGS. 3-4E, jaw liner 130 of jaw member 100, as mentioned above, is engaged with elongated distal portion 118 of structural body 110 and is positioned to oppose blade 32 (FIGS. 1-2B) such that blade 32 (FIGS. 1-2B) is inhibited from contacting structural body 110 of jaw member 100. Jaw liner 130 may be fabricated from a compliant material such as, for example, polytetrafluoroethylene (PTFE), such that blade 32 (FIGS. 1-2B) is permitted to vibrate while in contact with jaw liner 130 without damaging components of ultrasonic surgical instrument 10 (FIG. 1), e.g., structural body 110 of jaw member 100, and without compromising the hold on tissue clamped between jaw member 100 and blade 32 (FIGS. 1-2B). Other suitable materials are also contemplated. Jaw liner 130 may be monolithically or otherwise formed.

Jaw liner 130 of jaw member 100 includes an elongated body 132, a distal projection 134 extending distally from elongated body 132, and an elongated base 136 from which elongated body 132. Elongated body 132 defines a generally rectangular configuration having an at-rest width “WW2” that generally approximates the width “W2” of narrow body portion 120b of cavity 120 of structural body 110 of jaw member 100 to enable elongated body 132 to fit within narrow body portion 120b of cavity 120 with minimal play therebetween. Elongated body 132 defines a height greater than a height of narrow body portion 120b of cavity 120 such that elongated body 132 protrudes from cavity 120 and structural body 110, as best illustrated in FIG. 4C, although, in embodiments, elongated body 132 may be flush with, rather than protrude from, structural body 110. Elongated body 132 includes a tissue-contacting surface 133a that defines a plurality of teeth 133b. Teeth 133b inhibit slippage of tissue clamped between jaw member 100 and blade 32 (FIGS. 1-2B).

Distal projection 134 of jaw liner 130 extends distally from elongated body 132 and likewise defines a generally rectangular configuration. Distal projection 134 is configured for receipt within open distal neck 122 of cavity 120 of structural body 110 of jaw member 100.

Elongated base 136 of jaw liner 130 extends longitudinally along elongated body 132 of jaw liner 130 and defines an at-rest width “WW1” greater than the at-rest width “WW2” of elongated body 132 such that elongated base 136 overhangs elongated body 132 on either side thereof. As such, elongated body 132 and elongated base 136 define a generally T-shaped cross-sectional configuration. Elongated base 136 defines a height that generally approximates the height of relatively wide base portion 120a of cavity 120, while at-rest width “WW1” generally approximates the width “W1” of relatively wide base portion 120a of cavity 120. At-rest width “WW1,” more specifically, is slightly less than or equal to the width “W1” of relatively wide base portion 120a of cavity 120 but is greater than the reduced width “WR” of base portion 120a of cavity 120 defined at the location of each wedge 129.

Continuing with reference to FIGS. 3-4E, to engage jaw liner 130 with structural body 110, jaw liner 130 is slid distally through open proximal mouth 121 of cavity 120 of structural body 110 such that elongated body 132 enters body portion 120b of cavity and such that elongated base 136 enters base portion 120a of cavity 120. Jaw liner 130 is then slid distally through cavity 120 until distal projection 134 of jaw liner 130 is received within open distal neck 122 of cavity 120.

With jaw liner 130 received within cavity 120 of structural body 110, the T-shaped cross-sectional configuration of cavity 120 and corresponding T-shaped cross-sectional configuration of jaw liner 130 provide transverse retention (e.g., vertical and horizontal from the orientation illustrated in FIG. 4C) of jaw liner 130 within structural body 110. Longitudinal retention is provided, in part, via interference-fitting of elongated base 136 of jaw liner 130, having at-rest width “WW1,” within relatively wide base portion 120a of cavity 120, having width “W1.” However, this interference-fitting retention is insufficient on its own at least because it is necessarily minimal so as to enable the slidable insertion of jaw liner 130 into cavity 120 structural base 110.

The majority of longitudinal retention is provided via interference fit between jaw liner 130 and structural body 110 at the locations of wedges 129. More specifically, owing to the fact that the at-rest width “WW1” of elongated base 136 of jaw liner 130 is greater than the reduced width “WR” of base portion 120a of cavity 120 at the location of each wedge 129, wedges 129, in cooperation with the opposing first sidewall sections 127a, serve to compress the portion of the compliant jaw liner 130 disposed therebetween such that the width of compliant jaw liner 130 at the location of each wedge 129 is compressed to generally approximate the reduced width “WR,” thus providing interference-fit retention and maintaining jaw liner 130 in substantially fixed position relative to structural body 110. With wedges 129 spaced-apart along the length of cavity 120 and, in embodiments, staggered, the retention is provided along the lengths of jaw liner 130 and structural body 110 without compromising the ease of insertion of jaw liner 130 into cavity 120.

Further, with wedges 129 oriented such that the angled surfaces thereof are disposed in a generally proximally-facing orientation and such that the perpendicular surfaces thereof are disposed in a generally distally-facing orientation, wedges 129 facilitate locking jaw liner 130 in position. More specifically, while jaw liner 130 is more-easily cammed over the proximally-facing angled surfaces of wedges 129 as jaw liner 130 is translated distally through cavity 120, the distally-facing orientation of the perpendicular surfaces of wedges 129 inhibit camming of jaw liner 130 thereabout and, thus, inhibit proximal movement of jaw liner 130 relative to structural body 110. Distal movement is inhibited, once jaw liner 130 is fully inserted into cavity 120, by the abutment of the distal end of elongated base 136 of jaw liner 130 with distal lip 128 of structural body 110. Thus, distal lip 128, in conjunction with wedges 129, serve to lock jaw liner 130 in position, providing suitable retention without compromising ease of insertion.

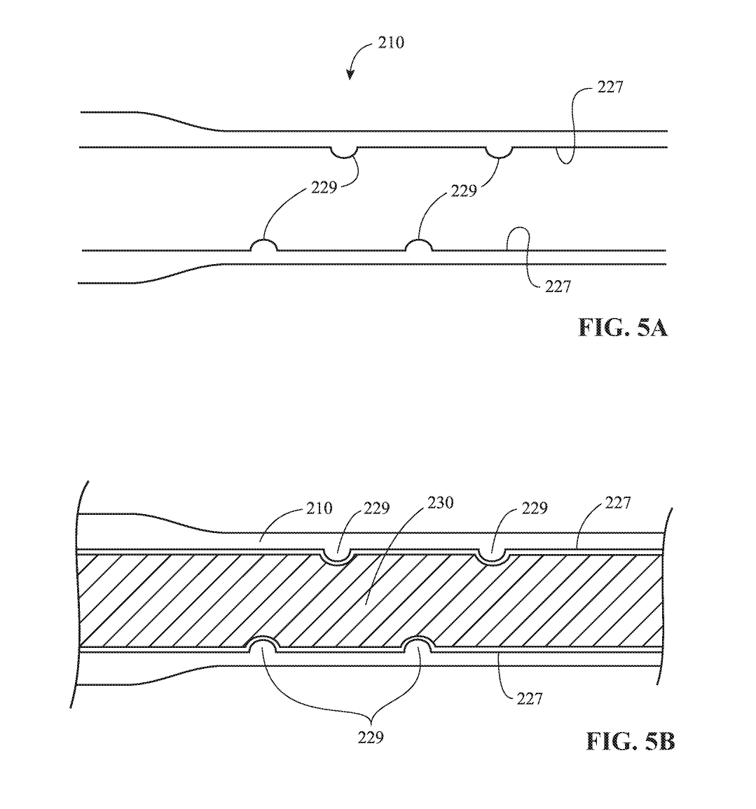

Turning now to FIGS. 5A-5B, another embodiment of a jaw member provided in accordance with the present disclosure and configured for use with ultrasonic surgical instrument 10 (FIG. 1), or any other suitable surgical instrument, is shown generally identified by reference numeral 200. Jaw member 200 includes a structural body 210 and a jaw liner 230 and is similar to and may include any of the features of jaw member 100 (FIGS. 1-4E), detailed above. Thus, only the differences between jaw member 200 and jaw member 100 (FIGS. 1-4E) are detailed below to avoid unnecessary repetition.

Jaw member 200 differs from jaw member 100 (FIGS. 1-4E) in that, rather than including engagement features in the form of wedges 129 (FIGS. 3-4B), the engagement features of structural body 210 of jaw member 200 are formed as humps 229. Humps 229, in cooperation with the opposing sidewall sections 227 of structural body 210, serve to compress the portion of the compliant jaw liner 230 disposed therebetween, thus providing interference-fit retention and maintaining jaw liner 230 in substantially fixed position relative to structural body 210. Other suitable configurations for the engagement features such as for example, other geometrical shapes or patterns, are also contemplated.

While several embodiments of the disclosure have been described above and illustrated in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A jaw member for a surgical instrument, the jaw member comprising:
a structural body including a proximal flange portion and an elongated distal portion extending distally from the proximal flange portion, the elongated distal portion including a floor and a pair of opposed sidewalls that cooperate to define a cavity having a first width extending between the opposed sidewalls, the elongated distal portion further including at least one engagement feature extending inwardly from one of the opposed sidewalls into the cavity such that the cavity defines a reduced width at a location of the at least one engagement feature; and
a jaw liner configured for slidable insertion into the cavity to engage the jaw liner with the structural body, the jaw liner defining an at-rest width that is less than or equal to the first width and greater than the reduced width such that, upon slidable insertion of the jaw liner into the cavity, the jaw liner is compressed at the location of the at least one engagement feature and retained therein via an interference-fit engagement.

2. The jaw member according to claim 1, wherein the cavity includes a relatively wide base portion and a relatively narrow body portion cooperating to define a generally T-shaped cross-sectional configuration of the cavity.

3. The jaw member according to claim 2, wherein the first width is defined within the relatively wide base portion of the cavity, and wherein the at least one engagement feature extends inwardly into the relatively wide base portion of the cavity.

4. The jaw member according to claim 2, wherein the jaw liner includes a relatively wide base portion and a relatively narrow body portion that cooperate to define a generally T-shaped cross-sectional configuration of the jaw liner.

5. The jaw member according to claim 1, wherein the jaw liner is formed from a compliant material.

6. The jaw member according to claim 1, wherein the cavity defined within the structural body includes an open proximal mouth to enable distal sliding of the jaw liner through the open proximal mouth and into a central portion of the cavity.

7. The jaw member according to claim 6, wherein the structural body includes a distal lip configured to inhibit further distal sliding of the jaw liner once the jaw liner is fully disposed within the cavity.

8. The jaw member according to claim 1, wherein the at least one engagement feature is a wedge.

9. The jaw member according to claim 8, wherein the wedge includes a proximally-facing angled surface and a distally-facing perpendicular surface.

10. The jaw member according to claim 1, wherein the at least one engagement feature is a hump.

11. The jaw member according to claim 1, wherein the at least one engagement feature includes a first plurality of spaced-apart engagement features extending inwardly from one of the opposed sidewalls into the cavity and a second plurality of spaced-apart engagement features extending inwardly from the other of the opposed sidewalls into the cavity.

12. The jaw member according to claim 11, wherein the first and second pluralities of spaced-apart engagement features are arranged in a staggered configuration relative to one another.

13. A surgical instrument, comprising:
a handle assembly;
an elongated body portion extending distally from the handle assembly; and
an end effector operably disposed at a distal end portion of the elongated body portion, the end effector including:
an energy-delivering component; and
a jaw member positioned to oppose the energy-delivering component and movable relative thereto between an open position and a closed position for clamping tissue between the jaw member and the energy-delivering component, the jaw member including:
a structural body including a proximal flange portion and an elongated distal portion extending distally from the proximal flange portion, the elongated distal portion including a floor and a pair of opposed sidewalls that cooperate to define a cavity having a first width extending between the opposed sidewalls, the elongated distal portion further including at least one engagement feature extending inwardly from one of the opposed sidewalls into the cavity such that the cavity defines a reduced width at a location of the at least one engagement feature; and
a jaw liner configured for slidable insertion into the cavity to engage the compliant jaw liner with the structural body, the jaw liner defining an at-rest width that is less than or equal to the first width and greater than the reduced width such that, upon slidable insertion of the jaw liner into the cavity, the jaw liner is compressed at the location of the at least one engagement feature and retained therein via an interference-fit engagement.

14. The surgical instrument according to claim 13, wherein the energy-delivering component is an ultrasonic blade and wherein the elongated body portion includes an ultrasonic waveguide extending therethrough, the ultrasonic waveguide configured to transmit ultrasonic energy to the ultrasonic blade.

15. The surgical instrument according to claim 14, wherein, in the closed position of the jaw member, the jaw liner is configured to oppose the ultrasonic blade.

16. The surgical instrument according to claim 13, wherein the cavity includes a relatively wide base portion and a relatively narrow body portion cooperating to define a generally T-shaped cross-sectional configuration of the cavity, wherein the first width is defined within the relatively wide base portion of the cavity, and wherein the at least one engagement feature extends inwardly into the relatively wide base portion of the cavity.

17. The surgical instrument according to claim 13, wherein the cavity defined within the structural body includes an open proximal mouth to enable distal sliding of the jaw liner through the open proximal mouth and into a central portion of the cavity, and wherein the structural body includes a distal lip configured to inhibit further distal sliding of the jaw liner once the jaw liner is fully disposed within the cavity.

18. The surgical instrument according to claim 13, wherein the at least one engagement feature is a wedge including a proximally-facing angled surface and a distally-facing perpendicular surface.

19. The surgical instrument according to claim 13, wherein the at least one engagement feature is a hump.

20. The surgical instrument according to claim 13, wherein the at least one engagement feature includes a first plurality of spaced-apart engagement features extending inwardly from one of the opposed sidewalls into the cavity and a second plurality of spaced-apart engagement features extending inwardly from the other of the opposed sidewalls, the first and second pluralities of spaced-apart engagement features arranged in a staggered configuration relative to one another.
